(19) United States
(12) Patent Application Publication
(10) Pub. No.: US 2008/0195084 A1
(43) Pub. Date: Aug. 14, 2008

(54) Medical Instrument Having Replaceable Tools and Method of Making Same

(75) Inventor: James A. Rinner, Franksville, WI (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(73) Assignee: PILLING WECK INCORPORATED

(21) Appl. No.: 11/883,719

(22) PCT Filed: Feb. 3, 2006
(86) PCT No.: PCT/US2006/004015

§ 371 Date: Apr. 3, 2008

Related U.S. Application Data
(63) Continuation of application No. 11/050,335, filed on Feb. 4, 2005, now abandoned.

(51) Int. Cl. A61B 17/00

(52) U.S. Cl. 606/1

(57) ABSTRACT

An instrument for use in medical surgery, and having a handle and a working tool extending along a common axis. A separable connection is between the handle and the tool, and the connection is of a polygonal shape in an axial view thereof. The connection is tapered for wedging together and has one portion on the handle and another portion on the tool, and there are planar surfaces on both portions but with fewer planar surfaces on the one portion compared to the number of planar surfaces on the other portion.

This invention relates to a medical instrument having replaceable tools, and a method of making the instrument. The replacement feature can be for substituting a more appropriate tool for a previous one but for performing the same function, or it can be for substituting a tool having a different function, all being relative to a base handle onto which the tools are mounted.

### BACKGROUND OF THE INVENTION

Instruments having the features of the foregoing paragraph are already known. The present invention improves upon that prior art by providing an instrument which is more precise, such as in its longitudinal alignment of the handle and the tool along a common axis. In that regard, the instrument of this invention provides a connection between the handle and the tool which is of an optimum stability and yet capable of being releasable for substituting tools. That is, there is no rocking of the tool radially of the instrument longitudinal axis, so the surgeon using the tool can apply the utmost accuracy in the manipulation of the tool on a patient.

This is a precision instrument for assuring the accurate placement of the working tool tip, for instance, and substitution tools also have that precision relative to the supporting handle. The instrument can be a curette which is useful in surgery. The instrument is arranged to effect easy, rapid, and assured accurate substitution of tools on the handle.

Further, the tools rotationally orientate with the handle and about the longitudinal axis of the entire instrument, and thereby substituted tools have the same rotated relationship relative to the handle as that of the previous tool, so, upon gripping the handle, the surgeon always knows the orientation of the tool working end.

A method of making the instrument is also included in this invention and is an improvement upon the prior art for the achievement of the foregoing features.

Other objects and advantages will become apparent upon reading the following description in light of the accompanying drawings.

### BRIEF DESCRIPTION OF THE DRAWINGS

FIG. 1 is a front perspective view of an instrument incorporating a preferred embodiment of this invention.

FIG. 2 is a perspective view like FIG. 1 but with the handle and the tool separated.

FIG. 3 is a top plan view of FIG. 1.

FIG. 4 is a sectional view taken on a plane designated by the line 4-4 in FIG. 3.

FIG. 5 is a side elevation view of FIG. 1.

FIG. 6 is a front elevation view of FIG. 15.

FIGS. 7, 8, and 9 are enlarged perspective views of three parts shown in FIG. 4.

FIG. 10 is an enlarged sectional view of a part shown in FIG. 4.

FIG. 11 is an enlarged perspective view of a part shown in FIG. 4.

FIG. 12 is an enlarged perspective view of a fragment of a part shown in FIG. 2.

FIG. 13 is an enlarged perspective view of a part shown in FIG. 4.

FIGS. 14, 15, and 16 are respectively top plan and side elevation an end elevation views of a part shown in FIG. 13.

FIG. 17 is an enlarged end elevation view of FIG. 15.

FIG. 18 is a section view taken on a plane designated by the line 18-18 in FIG. 17.

FIG. 19 is an enlarged view of the showing in the broken line circle of FIG. 4, on an enlarged scale.

### DETAILED DESCRIPTION OF A PREFERRED EMBODIMENT AND METHOD

The instrument of this invention is useful in the medical arts, particularly in surgery where the patient's body is penetrated by the instrument for probing and alterations of the body. It is herein shown as a curette which is capable of receiving and removing portions of the patient's body, such as tissue. That is, there is a handle and a tool separably connected with and supported by the handle, and various tools can be substituted for the original tool to present tools of different sizes or functions. In all instances, the assembled instrument with the handle and tool are in precision form, and the tool can be maneuvered by a surgeon in an optimum and most secure manner because the connection between the handle and the tool is arranged to simulate one solid instrument of handle and tool even though they are separable when desired.

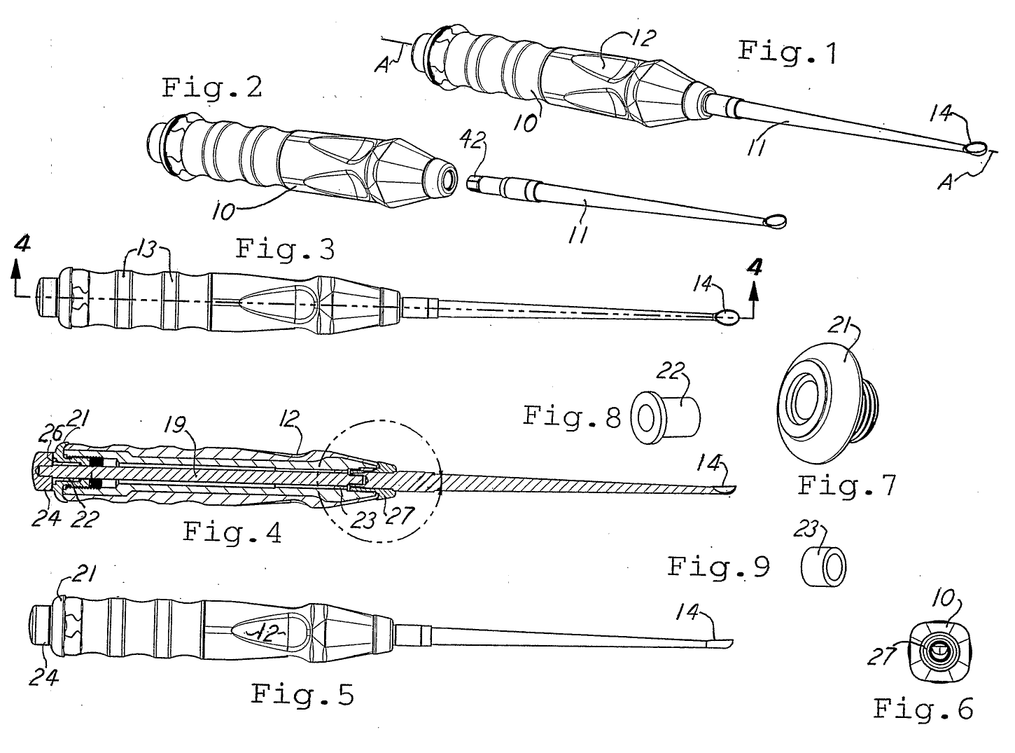

This instrument includes a handle 10 and a tool 11, both of which are elongated and extend coaxially along a common axis designated A-A in FIG. 1. FIG. 2 shows the handle 10 and the tool 11 separated from each other in the transitional state of being assembled or disassembled.

The drawings show that the handle 10 has a unique exterior configuration, including a thumb depression or surface 12 seen in FIG. 3. That is, the surgeon's fingers would be wrapped around the handle and its rings at 13, and the thumb would then naturally rest in the depression or on the surface 12, all in a natural positioning of the surgeon's fingers when holding the instrument in the operative mode. In that orientation, the tool has an upright portion 14 which will be disposed in the upright or upwardly facing orientation, as seen in FIGS. 1, 2, and 3. In that relationship, the surgeon will always know the upright and proper positioning of the operative end though the surgeon can not actually see the end 14 which may be in the patient's body. The end 14 is like a spoon for handling material.

The hereinafter description reveals the connection between the handle 10 and the tool 11 for achieving the upright relationship mentioned above. That connection is polygonal in configuration when viewed along the axis A, with a shown female portion on the handle 10 and a male portion on the tool 11. However, it will be apparent the female and male relationship could be interchanged between the handle and the tool.

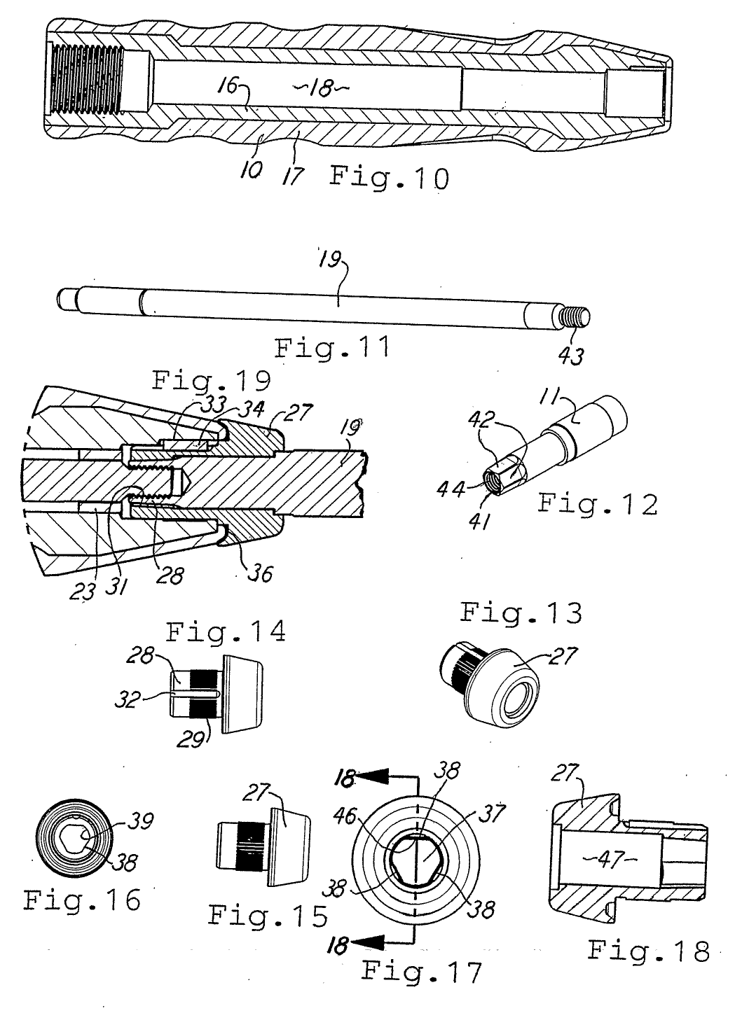

As shown in FIG. 10, the handle 10 has a base 16 of a rigid metal material and there is a cover 17 encapsulating the base 16. The cover 17 can be of an elastomer material for gripping and control. A hollow interior 18 extends the length of the handle 10, and a rod 19 is disposed in the handle interior 18. A cylindrical member 21 is shown threaded into the handle 10 and it supports a bushing 22 which is best shown in FIG. 8.

Also, there is a bushing 23 in the handle 10, so the bushings 22 and 23 are respectively disposed at opposite ends of the handle 10 in spaced-apart relationship. Those cylindrical bushings 22 and 23 are thusly supported by the handle 10 and they rotatably support the rod 19 in the handle 10 and on the axis A. The rod 19 can move axially along the axis for a purpose mentioned later. A button 24 is affixed to the rod 19 for inducing the rotation and axial movement of the rod 19, and the button 24 abuts the member 21 at 26 in the rightward movement of the assembled rod 19 and button 24, such as shown in FIG. 4, to thereby limit rightward movement of the rod 19.

The handle 10 includes a member 27, shown in the shape of a sleeve and having a cylindrical extension 28, and member 27 is affixed to the handle 10. The shown arrangement for affixing is that of splines 29 which are snug with the handle cylindrical interior at 31. Thus, in any conventional manner, the member 27 is a part of and is affixed with the handle 10. Also, the extension 28 has a slit 32 and the handle has a slot 33, and the slit and the slot mutually receive a pin 34 and that rotationally aligns the member 27 with the remainder of the handle 10.

The handle 10 and the member 27 axially abut each other at 36, so the member 27 is axially limited relative to the remainder of the handle 10. With elasticity in the cover 17 which intervenes between the handle core 16 and the member 27, the member 27 can actually be pressed against the cover at 36 for maximum axial securement of a hereinafter described wedging connection between the tool 11 and the handle 10.

The hollow interior 37 of the member 27 is formed to present three planar surfaces 38 which are equally spaced around, and radially from, the axis A. Between every two surfaces 38, the interior 37 has relieves at 39. Thus, the surfaces 38 present the radially closest surfaces to the axis A on the interior of the member 27. The three surfaces 38 are in triangular configuration affixed on the handle 10.

The end of the rod 19 has a hexagonal shape in the axial view thereof, as seen at 41 in FIG. 12, and it presents planar surfaces 42. The six surfaces 42 are all of the same shape and are contiguous in their disposition around the axis A. As seen in FIG. 19, the rod 19 is inserted into the interior 37 of the member 27, and the three surfaces 38 are in contact with and thus mate with a respective three of the six surfaces 42. In that mating, only three sides of the hexagon at 41 contact the member 27 and thus optimum stability is achieved in the male and female mating of the handle 10 and the tool 11. There is no rocking radially of the axis A of the tool 11 relative to the handle 10.

It will be apparent that the location of the hexagon and the triangle could be interchanged relative to the handle 10 and the tool 11, with the hexagon being on either the handle or the tool, and likewise with the triangle. The connection is of a polygonal shape in that it is only of planar surfaces in contact with each other.

Those mating surfaces 38 and 42 are disposed on a tapered angle shown at 47 in FIG. 18, and they are relative to the axis A, so the surfaces wedge together upon axial movement of the rod 19 into the handle 10. That movement is induced by having the rod 19 threaded at 43 and the interior of the tool likewise is threaded at 44. Then, upon rotation of the rod 19, the tool 11 is drawn into secure contact with the handle by having the mating surfaces 38 and 42 wedge together for rendering the tool 11 virtually solid relative to the handle 10. The relief portions at 38 are always radially spaced from any part of the tool hexagon, so they play no role in the connection between the handle 10 and the tool 11.

Upon rotating the rod 19 counterclockwise, it backs away from the tool 11 and the tool can then be released and removed from the handle and another tool can be inserted, if desired. So the handle 10 has a rotational orientation marking, such as the surface 12, to indicate its upright orientation. One of the hex surfaces 42 is orientated to align with the upright portion 14, as shown in FIG. 2. Then the surgeon can insert the tool 11 with its portion 14 facing upright, and thereby the surgeon will have the portion upright whenever the handle is upright per its marking. The triangular surfaces accommodate the upright relationship in that they are orientated according to the handle marking 12 and thus will mate with a respective three of the surfaces 42, all to have the handle marking 12 and the tool upright at 14 aligned with each other. The surgeon will then always know the orientation of the portion 14 when it is inside the patient's body.

The method of making the instrument is inherently disclosed in the foregoing. Additionally, the triangular configuration can be formed by forming the hole in the member 27 of say a radius of a distance from the axis A to the nearest point on the surfaces 38, and that is at 46 in FIG. 17. Then an electrode can be inserted into the hole to form the surfaces 38 and the relieves 39 to achieve the final triangular configuration shown in FIG. 17, with the taper mentioned.

While specific constructions and methods are disclosed herein, it will be understood that changes can be made therein and still be within the scope of this invention.

What is claimed is:

1. A medical instrument having a handle and operative tool with both thereof disposed along an elongated axis and being joined together with a separable connection therebetween and a threaded rod extending between said handle and said tool for releasably securing said handle and said tool together at said connection by drawing said handle and said tool toward each other along said axis, the improvement comprising:
said connection being in a polygonal shape in a view of said connection along said axis and said polygonal shape presenting planar surfaces on both said handle and said tool and with respective ones of said planar surfaces being in mating contact with each other and with said planar surfaces on a first one of said handle and said tool being in a total number different from the total number on a second one of said handle and said tool, and
all said planar surfaces being tapered relative to said axis for wedging said handle and said tool together along said axis upon tightening said rod relative to said handle and said tool for drawing said handle and said tool toward each other along said axis.

2. The medical instrument as claimed in claim 1, wherein:
the polygonal shape of said first one of said handle and said tool is a hexagonal shape.

3. The medical instrument as claimed in claim 2, wherein:
the polygonal shape of said second one of said handle and said tool is in a total number of three planar surfaces.

4. The medical instrument as claimed in claim 3, wherein:
said three planar surfaces are equally spaced apart around said axis for respectively contacting three surfaces of said hexagonal shape.

5. The medical instrument as claimed in claim 1, wherein:
said first one of said handle and said tool is that of said tool itself.

6. The medical instrument as claimed in claim 5, including:
said connection is that of a male portion and female a portion and said tool includes said male portion.

7. The medical instrument as claimed in claim 1, including:
said connection includes a sleeve coaxial with said axis and being affixed with and a part of said handle and presenting said planar surfaces of said handle.

8. The medical instrument as claimed in claim 7, including:
a rotational orientation member interposed between said sleeve and said handle for disposing said planar surfaces of said handle in a selected rotational orientation about said axis and relative to the remainder of said handle, and thereby orientate said tool in a rotated orientation about said axis and relative to said axis.

9. The medical instrument as claimed in claim 7, including:
a spline connection between said sleeve and the remainder of said handle for affixing said sleeve with and as a part of said handle.

10. The medical instrument as claimed in claim 1, including:
there being a plurality of said tools and each one of said tools having a working portion with an upright orientation for performing work,
said handle having a hand-grip surface rotationally orientated about said axis and relative to said upright orientation of said tool for presenting said tool working portion in the upright orientation upon gripping said handle on said hand-grip surface, and
said planar surfaces being orientated about said axis for presenting said working portion of each of said tools in said upright orientation in the mating contact of said planar surfaces.

11. The medical instrument as claimed in claim 1, including:
said handle having bushings spaced apart along said axis for rotationally supporting and radially aligning said rod relative to said axis.

12. A medical instrument having a handle and an operative tool with both thereof disposed along an elongated axis and being joined together with a separable connection therebetween and a threaded rod extending between said handle and said tool for releasably securing said handle and said tool together at said connection by drawing said handle and said tool toward each other along said axis, the improvement comprising:
said connection having a handle portion and a tool portion and said tool portion including six planar surfaces to form a hexagonal shape on said tool in a view thereof along said axis and said handle portion including a total of only three planar surfaces on said handle for respectively mating with three of said six planar surfaces.

13. The medical instrument as claimed in claim 12, wherein:
all said planar surfaces are tapered relative to said axis for wedging said handle and said tool together along said axis upon tightening said rod relative to said handle and said tool for drawing said handle and said tool toward each other along said axis.

14. The medical instrument as claimed in claim 13, wherein:
said three planar surfaces are equally spaced apart around said axis for respectively contacting three surfaces of said hexagonal shape.

15. The medical instrument as claimed in claim 12, wherein:
said three planar surfaces are equally spaced apart around said axis for respectively contacting three surfaces of said hexagonal shape.

16. A method of making a medical instrument having a handle and an operative tool with both thereof disposed along an elongated axis and being joined together with a separable connection therebetween of a male portion and a female portion and said instrument having a threaded rod extending between said handle and said tool for releasably securing said handle and said tool together at said connection by drawing said handle and said tool toward each other along said axis, comprising the steps of:
forming said connection to present on a first one of said handle and said tool a polygonal shape in a view thereof along said axis and having said polygonal shape include planar surfaces on each of said handle and said tool and with said planar surfaces disposed in mating contact with each other and with said planar surfaces on said first one of said handle and said tool being in a total number different from the total number of said planar surfaces on a second one of said handle and said tool, and
forming said female portion of said connection by applying an electrode for shaping said female portion and its said planar surfaces.

17. The method of making a medical instrument, as claimed in claim 16, comprising the step of:
forming a total number of only three planar surfaces on the first one of said handle and said tool and forming a total number of six planar surfaces on the second one of said handle and said tool.

18. The method of making a medical instrument, as claimed in claim 17, comprising the step of:
forming all said planar surfaces on an angle tapered relative to said axis and wedging said handle and said tool together along said axis upon tightening said rod relative to said handle and said tool for drawing said handle and said tool toward each other along said axis.

19. The method of making a medical instrument, as claimed in claim 18, comprising the step of:
rotationally aligning a gripping surface on said handle and said about said axis with said planar surfaces for replacement of said tool with another tool, and
placing and connecting a second one of said tool relative to said handle.
